(19) United States
(12) Patent Application Publication
(10) Pub. No.: US 2023/0057579 A1
(43) Pub. Date: Feb. 23, 2023

(54) CARDIOVASCULAR DEVICE AND KIT FOR THE REDUCTION OF A CARDIAC CAVITY

(71) Applicant: EUCARDIA S.R.L., Milano (IT)

(72) Inventors: Roberto Parravicini, Modena (IT); Marco Bottaro, San Mauro Torinese (IT); James Leo Pokorney, Northfield, MN (US)

(73) Assignee: EUCARDIA S.R.L., Milano (IT)

(21) Appl. No.: 17/794,751

(22) PCT Filed: Jan. 25, 2021
(86) PCT No.: PCT/IT2021/050022

§ 371 Date: Jul. 22, 2022

(30) Foreign Application Priority Data
Jan. 23, 2020 (IT) 102020000001252

(51) Int. Cl. A61M 60/148; A61M 60/295; A61M 60/89

(52) U.S. Cl. CPC A61M 60/148; A61M 60/295; A61M 60/89; A61M 60/497

(57) ABSTRACT

The cardiovascular device (1) comprises a diaphragm assembly designed to be inserted into a ventricular cavity (VS) substantially transverse in order to reduce its volume, said diaphragm assembly having a peripheral edge (2B) that can be sealingly engaged on the walls (5) of the cavity 7 and being alternately driven between an active blood thrust position and an inactive position, said assembly being at least partially deformable in response to contractions of the walls (5) and comprising a balloon-shaped elastic body (2; 100; 200; 300; 400) which has an external surface (2A; 103) that defines and encloses an internal cavity (CI; CI2) and which can be configured between a gathered position of minimum bulk, an everted position of maximum bulk and vice-versa, at least one mobile portion (3; 3′) of the peripheral surface which is disposed transverse/diagonal and which is surrounded by the peripheral edge and at least one aperture (3A) for access from the outside to the internal cavity (CI; CI2).

### FIELD OF THE INVENTION

The invention concerns a cardiovascular device and a kit for the reduction of a cardiac cavity, generally usable to compensate for cardio-dilation pathologies that affect the ventricles of the heart muscle, in particular cardio-dilation pathologies of the left ventricle.

### BACKGROUND OF THE INVENTION

Cardio-vascular devices are known, which are implanted in patients suffering from cardio-dilated disease which reduces their ability to pump blood in the circulatory system, through the aorta, to reduce the internal volume of a ventricle of the heart, typically the left ventricle.

Typically, known cardio-vascular devices consist of an elastic diaphragm which is implanted inside the ventricle in a transverse position with respect to the greater longitudinal size of the latter and which is supported by a reticular micro-frame which is also elastically deformable, in such a manner that, after implantation, these devices can decrease the functional volume of the ventricle to make it perform the function of pumping blood that it normally performs in normal conditions, that is, in the absence of pathologies.

The micro-frame is generally made in the form of a sort of small umbrella which has a central hub from which a series of elastic arms extend in a radial direction and on which the diaphragm, typically in the form of a membrane, rests extended and to which it is attached.

The overall elasticity of these cardio-vascular devices allows them to follow the systole and diastole movements of the heart and to accompany them, alternately flexing out in the ventricle during the systolic phase and retracting into a distended position during the diastolic phase, or, on the contrary, flexing in the diastolic phase and retracting in the systolic phase, according to the use for which they are made.

In short, these diaphragms behave like a micro-pump that sucks blood from the left atrium and pushes it toward the circulatory system through the aorta.

In order to perform their function, these cardio-vascular devices have to maintain perimetrically a hermetically sealed contact with the internal walls of the ventricle and to obtain this sealed contact, the diaphragm can be equipped with a peripheral ring which, in addition to maintaining the support against the internal surface of the walls of the ventricle, joins the ends of the arms together.

In order to make the position of the devices stable the ends of the arms are pointed, so as to penetrate stably inside the organic tissue of the walls of the ventricle and are clamped to this, possibly also using sutures.

The implantation of these devices inside the ventricle is normally performed surgically, through an incision made in the lower apical part of the heart.

According to another technique, implantation takes place by introducing the devices percutaneously, that is, by using the passage inside a large-caliber blood vessel, for example the femoral artery, until it reaches the ventricle of the heart, typically the left ventricle.

In both cases, the devices are previously prepared in a folded configuration of minimum bulk, and inserted inside an introducer catheter which, after the implantation zone has been reached, is retracted by the operating surgeon or cardiologist, leaving the devices free to open and assume their normal position of use in the ventricle.

From patent U.S. Pat. No. 5,139,517 an orthotopic intraventricular cardiac pump is known, which is able to increase the volumes of one or both ventricles of the heart.

The pump comprises a diaphragm which is hydraulically driven by an automatic physiological pumping system associated with a pacemaker which is powered by rechargeable batteries.

Patent US2007/161846 describes a device and a method to improve the diastolic function of the heart which comprises a central element from which extend in a radial direction a multiplicity of splints on which a membrane is extended and constrained.

The central element and the splints form a flexible concave structure that is intended to be positioned transversely inside a ventricle, typically the left ventricle of the heart, and which is able to elastically follow the systole and diastole movements thereof.

This structure, according to the patent, is suitable to improve cardiac function specifically in the diastole phase, that is, in the phase of recalling the blood inside the ventricle, although the patent provides a possible location and use in other cardiac cavities.

This device has two configurations of use as well, that is, a folded configuration of minimum bulk so that it can be placed inside a cardiac cavity by means of an introducer catheter, or surgically, and an extended operating configuration in which it occupies an apical part of the internal volume of the cardiac cavity.

This state of the art has some disadvantages.

One disadvantage is that known cardiovascular devices must be attached to the internal walls of the cardiac cavities, often by means of sutures, in order not to change their position during functioning, and this implies that the walls of the cardiac cavities are passed through by the sutures also in zones affected by pathologies that reduce their thickness and consistency.

Another disadvantage is that the ends of the arms or splints must penetrate at least for a certain distance into the walls of the cardiac cavities in order to guarantee both the stability and the hermetic seal of the diaphragms which divide the internal volume into two parts, one active or functional with respect to the circulating volumes of blood, and one inactive because it is excluded from the hemodynamic activity.

Another disadvantage is that it is not possible to regulate the functioning characteristics of the cardiovascular devices as a function of the pressure inside the ventricles.

Another disadvantage is that known cardiovascular devices have to be made according to the specific profile of the cardiac cavity for which they are intended.

### PURPOSES OF THE INVENTION

The purpose of the invention is to overcome the disadvantages described above, providing a cardiovascular device and a kit for the reduction of a cardiac cavity which allow to divide its volume in an adjustable manner and to simultaneously maintain functionality even in the presence of dilation pathologies.

Another purpose of the invention is to produce a cardiovascular device and a kit for the reduction of a cardiac cavity that allow to adjust the dimensions and conformation of the device even after implantation on site.

Another purpose of the invention is to produce a cardiovascular device and a kit for the reduction of a cardiac cavity that can be positioned on site both percutaneously and also surgically.

According to one aspect of the invention, a cardiovascular device is provided, in accordance with the characteristics of claim 1.

According to another aspect of the invention, a kit for the reduction of a cardiac cavity is provided, in accordance with the characteristics of claim 6.

The invention allows to obtain the following advantages:

maintain ventricular function even in the presence of a dilation pathology;

regulate the structural and functional characteristics of the cardiovascular device as a function of the characteristics of conformation and condition of the cardiac cavity;

standardize the production of the cardiovascular device, reducing its overall costs;

limit the need for traumatic attachments between the cardiovascular device and the walls of the cardiac cavity in which it is implanted.

### BRIEF DESCRIPTION OF THE DRAWINGS

Other characteristics and advantages of the invention will become more apparent from the following description of preferred, but not exclusive, embodiments of a cardiovascular device and a kit for the reduction of a cardiac cavity, shown by way of a non-limiting example in the attached drawings wherein:

FIG. 1 is a schematic section view of a heart, in the left ventricle of which there is implanted a cardiovascular device according to the invention;

FIG. 2 is an enlarged schematic view of a left ventricle in which the cardiovascular device is implanted, in a systole phase;

FIG. 3 is an enlarged schematic view of the left ventricle of FIG. 1, in a diastole phase;

FIGS. 4A to 4D schematically show the implantation steps of the cardiovascular device according to the invention, in a first embodiment version intended for percutaneous implantation;

FIG. 5 is a schematic cross-section and enlarged scale view of the left ventricle of FIG. 1 in which the cardiovascular device according to the invention is implanted, in a second possible embodiment intended for surgical implantation;

FIG. 6 is a schematic view of the ventricle of FIG. 5, in a systole phase;

FIG. 7 is a schematic view of the ventricle of FIG. 5, in a diastole phase;

FIGS. 8A to 8E schematically show the implantation steps of the cardiovascular device according to the invention, in the second embodiment version intended for transapical implantation;

FIG. 9 is a schematic view of a device for positioning the cardiovascular device according to the invention, in a configuration of insertion inside a ventricle;

FIG. 10 is a partially transparent view of the positioning device of FIG. 9;

FIG. 11 is a schematic and partially exploded view of the cardiovascular device according to the invention in the second embodiment version;

FIG. 12 is an interrupted detailed view on an enlarged scale of a part of the cardiovascular device according to the invention;

FIG. 13 is a very schematic view of the external profile of the cardiovascular device according to the invention in an inactive configuration of diastole of the heart and in which the profile that the device assumes in the systole phase is indicated with dashed lines;

FIG. 14 is a view of the cardiovascular device according to the invention in another possible version;

FIG. 15 is a lateral view of the cardiovascular device of FIG. 14;

FIG. 16 is a schematic view showing an implantation step of the cardiovascular device of FIG. 14;

FIG. 17 is a schematic longitudinal section view of an embodiment of an expandable balloon that forms another version of the cardiovascular device according to the invention, in a condition of cardiac systole;

FIG. 18 is a view of the balloon of FIG. 17, in a condition of cardiac diastole;

FIG. 19 is a schematic view on an enlarged scale of a detail of FIG. 17;

FIG. 20 is a schematic longitudinal section view of an expandable balloon that forms another embodiment version of the cardiovascular device according to the invention, in a condition of cardiac systole;

FIG. 21 is a schematic longitudinal section view of an expandable balloon which forms another embodiment version of the cardiovascular device according to the invention, in a condition of cardiac diastole;

FIG. 22 is a very schematic transparent view of another embodiment of an expandable balloon for the cardiovascular device according to the invention;

FIG. 23 is a cross-section view of FIG. 22, taken according to a plane XXIII-XXIII;

FIG. 24 is a very schematic transparent view of another embodiment version of an expandable balloon for the cardiovascular device according to the invention;

FIG. 25 is a schematic cross-section view of the expandable balloon of FIG. 24, taken according to a plane XXIV-XXIV.

### DETAILED DESCRIPTION OF A PREFERRED EXAMPLE EMBODIMENT

With reference to the drawings above, number 1 indicates as a whole a cardiovascular device, hereafter briefly device 1, intended for implantation in a cardiac cavity which has a greater longitudinal dimension and a smaller transverse dimension, for example a left ventricle VS of a heart C of a patient with ventricular dilated heart disease.

The person of skill understands that the device 1 can also be implanted in other cardiac cavities, such as, as a non-limiting example, the right ventricle VD, or the left atrium AS or even the right atrium AD.

The device 1 can also be implanted in any other biological cavity whatsoever, the overall volume of which it is necessary to reduce in order to perform therapies.

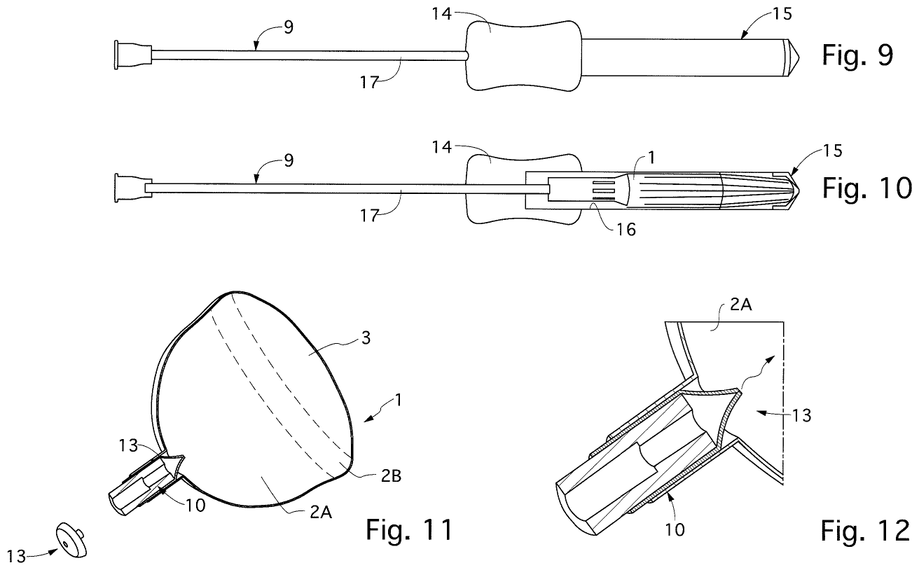

In detail, the device 1, which on the whole forms a diaphragm assembly, comprises a balloon-shaped body 2 which defines an internal chamber CI and which can assume two configurations, typically a folded configuration for introduction into the left ventricle VS, suitable for the implant to be perfornied and visible in detail in FIG. 10, and a dilated (or open) configuration of use, in which it occupies part of the left ventricle VS, reducing its internal dimensions.

In detail, it can be noted that the body 2 in the normal dilated configuration of use consists of a substantially semi-spherical cap portion, indicated with 2A, and a peripheral edge 2B, slightly everted with respect to the cap portion 2A, which surrounds it and delimits a portion 3 mobile according to the directions indicated by the arrows F1 and F2, along the greater dimension of the left ventricle VS.

The cap portion 2A is conformed to be able to rest, self-adapting, on the bottom 4 of the left ventricle VS and on the internal wall 5 thereof, while the peripheral edge 2B, which is radially everted with respect to the cap portion 2A, adheres to the internal wall 5 of the ventricle, creating therewith a hermetically sealed peripheral rest coupling.

The body 2 and the edge 2B are advantageously made of elastically flexible material, typically a polymer, which is biologically compatible with the organic tissues of the heart C and which allows the device 1 to flex and stretch freely in response to contractions of the left ventricle VS during the systole and diastole phases.

As can be seen in the drawings, in particular in the systole phase, the mobile portion 3 is everted toward the inside of the left ventricle VS while, in the diastole phase, it is released in the direction of the bottom 4.

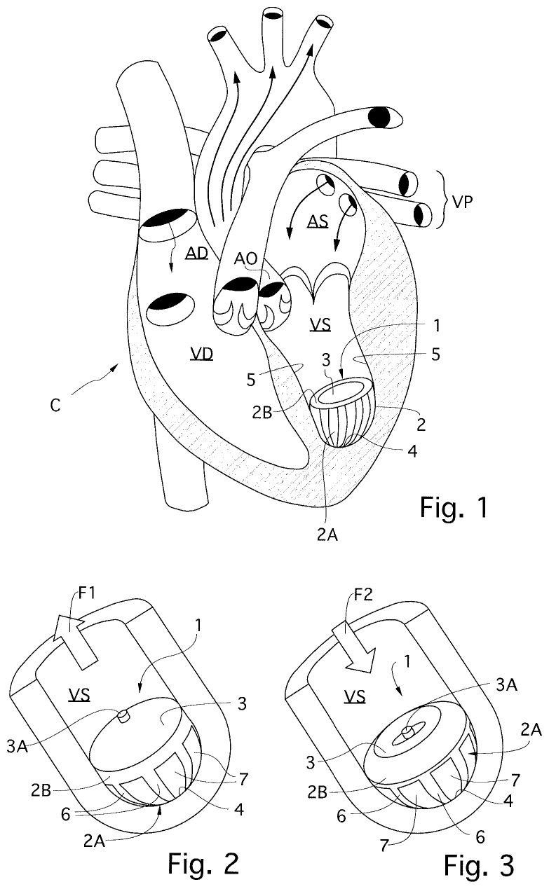

Therefore, the mobile portion 3 behaves like a pump which during the diastole phase, considered inactive, creates a suction of the blood coming from the left atrium indicated by VP and which accumulates inside the left ventricle VS, while, in the systole phase, considered an active phase, the mobile portion 3 is everted and thrusts the blood drawn during the diastole toward the aorta indicated in FIG. 1 with AO.

When the device 1 is implanted in the left ventricle VS, it occupies a part of its overall volume, reducing it, that is, reducing its extension, and becoming an artificial prosthesis that restores the substantially original functional conditions of the heart in it, even in the presence of cardio-dilation pathologies.

The cap portion 2A comprises a series of stiffening elements which are preferably disposed in a radial pattern, indicated with reference 6, and which delimit wedge-shaped zones 7 between them which have an elasticity, and therefore a flexibility, greater than that of the stiffening elements 6.

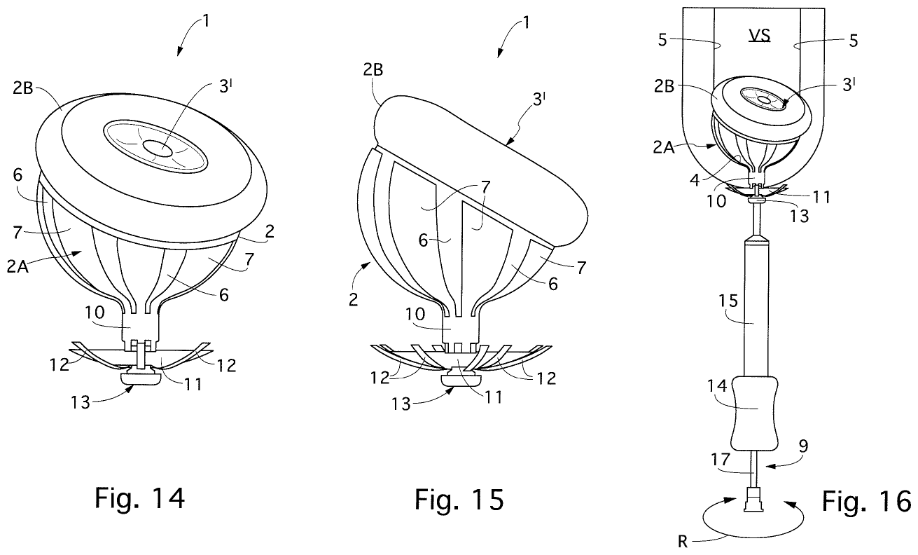

With reference to FIGS. 14-16, it can be noted that the device 1 can be made, if necessary, with the mobile portion 3 disposed inclined and indicated in this case with 3′.

This specific characteristic allows to position the device 1 more accurately as a function of the morphological characteristics of the left ventricle VS of each patient, possibly rotating it during the positioning, as indicated schematically in FIG. 16 with the arrow R, in order to find the best position for it to perform its function.

It should be noted that in order to expand the device 1 from the folded configuration of introduction to that of use, the internal cavity C1 of the body 2 is filled with a fluid, for example a saline solution, which is injected through a specific aperture, described below, by means of a syringe 8 (or equivalent device) and a catheter 9 which is also used to introduce the device 1 into the left ventricle VS.

In particular, the injection of the saline solution makes the edge 2B malleable and self-adaptable to the surface of the internal wall 5 in order to follow its profile in a mating manner and make a hermetically sealed contact with it.

As can be seen in the drawings, two versions of the device 1 can be made, namely one version for percutaneous implantation and one version for surgical implantation.

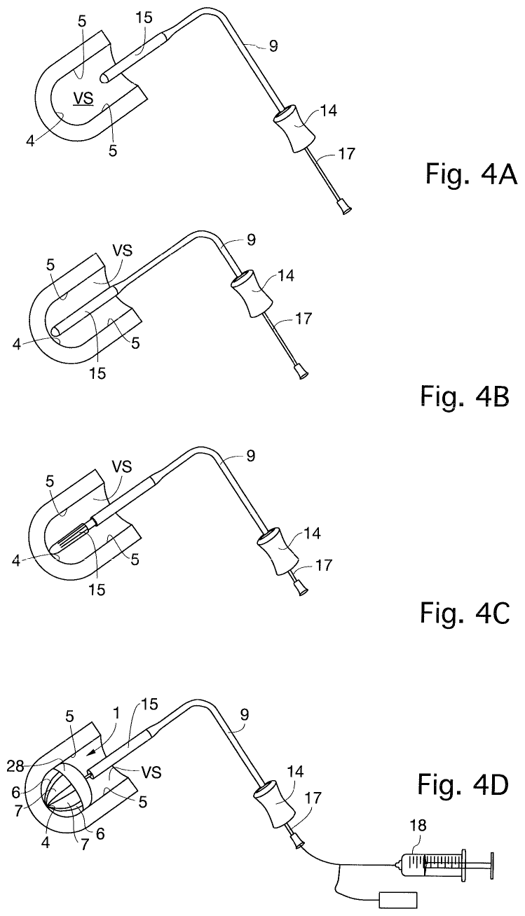

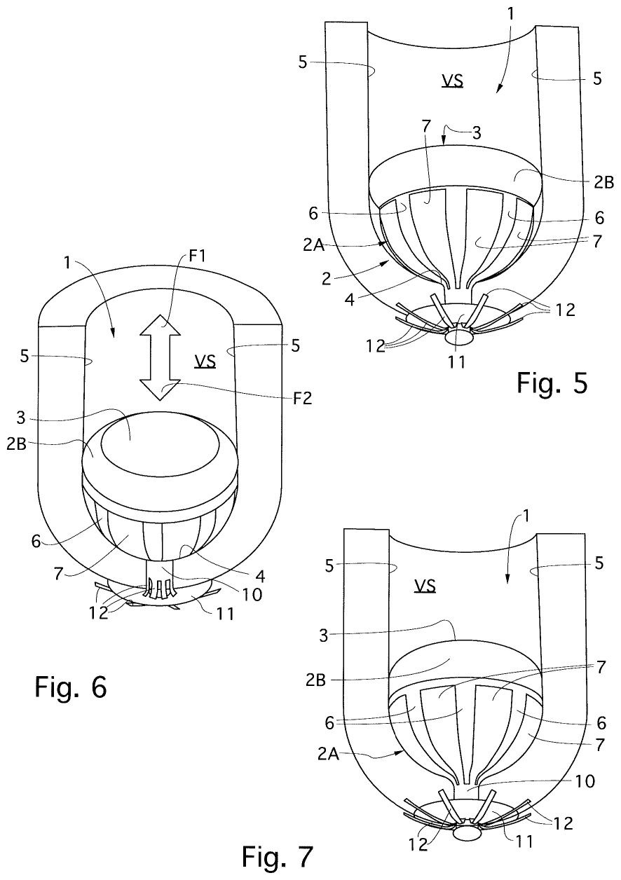

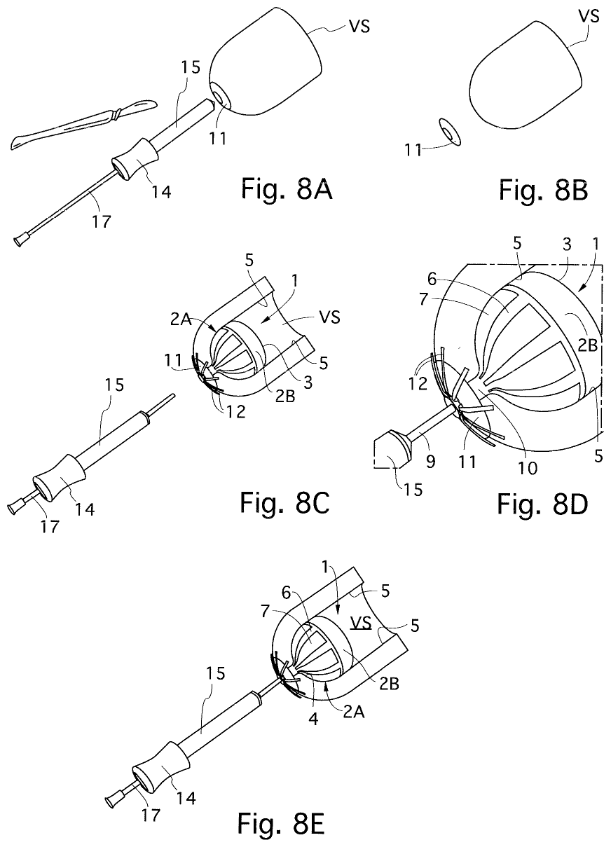

In the first case, which can be seen in FIGS. 2, 3 and 4A to 4D, the body 2 has an aperture 3A in the mobile portion 3 to allow access to the internal cavity CI by means of the catheter 9, while in the second case, which can be seen in FIGS. 5, 6, 7, 8A to 8E, 11, 12, 14, 15, 16, the body 2 has a segment of duct 10 which extends from the apical end of the body 2 opposite the portion 3 (or 3′) and which has a length sufficient to pass through the thickness of the biological tissue of the heart C in the corresponding apical zone of the bottom 4 of the left ventricle VS, protruding outside of the latter.

In this second case, on the end facing the outside of this segment of duct 10, there can be attached a stop split pin 11, provided to tighten between it and the body 2 the thickness of the tissue of the apical zone of the left ventricle VS.

In a preferred embodiment, the segment of duct 10 is equipped with a series of radial elements 12 which can be folded radially after passing through the split pin 11 in order to tighten it against the body 2.

In both versions, both the aperture 3A and also the free end of the segment of duct 10 are controlled with valve means 13 which can alternatively assume an open or closed configuration, according to requirements and, for example, can be manufactured as a one-way valve of the so-called “flute beak” type which allows a flow of saline solution to pass toward the internal chamber CI, but prevents it from refluxing in the opposite direction.

According to the invention, a kit of specific components is provided to implant the device 1 in the left ventricle VS.

In order to implant the device 1 percutaneously, the kit comprises the catheter 9, the syringe 8 and a handpiece 14 fitted on the catheter 9 to perform the implantation maneuvers.

In detail, the catheter 9 is equipped with a tip 15 which inside it has an axial cavity 16 inside which the device 1 is accommodated in the folded configuration of minimum bulk, designed for the introduction into the left ventricle VS and the implantation, as can be seen in detail in FIG. 10.

The catheter 9 also comprises a plunger 17 which is used by the surgeon to thrust the device 1 out of the axial cavity 16 after the tip 15 has been positioned inside the left ventricle VS, using the passage through a large caliber blood vessel of a patient, for example the femoral artery.

In order to implant the device 1 surgically, the kit is substantially the same as the one described for percutaneous implantation, to which the split pin 11 and the radial elements 12 are added.

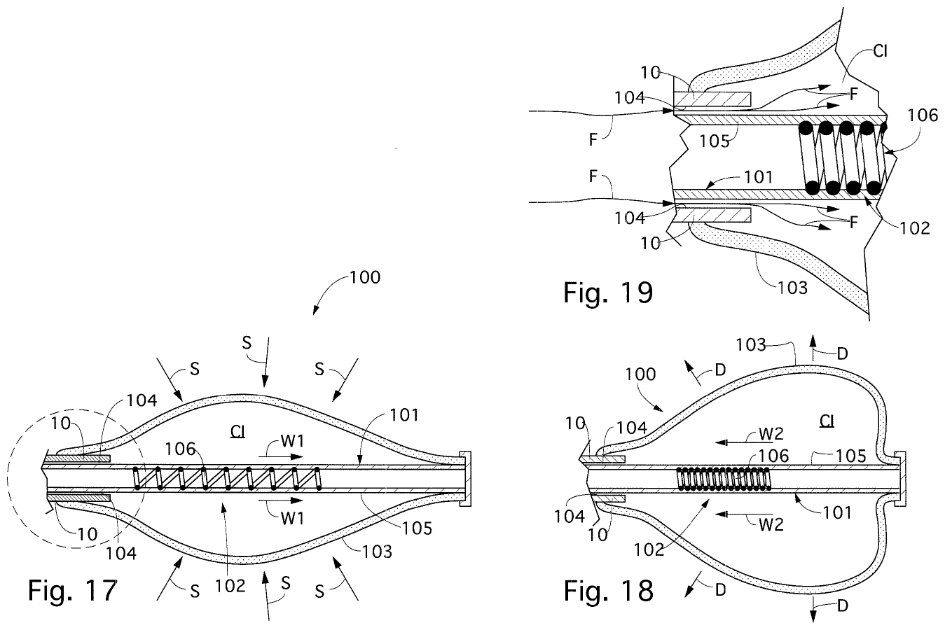

With reference to FIGS. 17 and 18, in which for simplicity identical reference numbers correspond to elements that are identical to the other drawings 1-16, 100 indicates an expandable or contractable balloon-shaped body, which comprises an internal core 101 which is housed in the internal chamber CI defined in the balloon-shaped body 100 and which has an axial length that can be modified elastically with modification means 102.

The balloon-shaped body 100 has an expandable external surface 103 which is kept in contact with the internal wall 5 of the left ventricle VS, in such a way as to elastically follow the systole and diastole movements of the same ventricle VS, occupying the opposite part thereof with respect to the left atrium AS.

The internal chamber CI is expandable by introducing a fluid F, for example a saline solution, which can be injected through an annular passage 104 which is defined between the segment of duct 10 and the internal core 101 and which is driven by the valve means 13 (not visible in the drawings).

The internal core 101, in this specific embodiment, consists of a tubular element 105 made of a biocompatible plastic material, very elastic and very thin, which incorporates, preferably inside it, but possibly also on the outside, a spring element 106, able to be loaded axially, which forms the modification means 102 and which is able to allow the variations in length of the tubular element 105 during the transitions between the systole and diastole phases of the left ventricle VS, as schematically indicated with the arrows W1 (systole) and W2 (diastole).

The spring element 106, in one variant, can also be interposed between the segment of duct 10 and the tubular body 101.

Overall, the spring element 106 fulfills a role similar to that of a lung capable of accumulating the thrust of the balloon-shaped body 100 during the systole and diastole phases of the heart C.

It should be noted that the ends of the balloon-shaped body 100 are attached in a sealed manner respectively on the segment of duct 10 and on the tubular body 101 with known attachment means, for example with gluing means, however the person of skill can imagine using any other attachment mean whatsoever available in the specific sector.

In FIGS. 17 and 18 the arrows S and D indicate the systole and diastole movements of the left ventricle LV in which the cardiovascular device 100 is intended to be implanted.

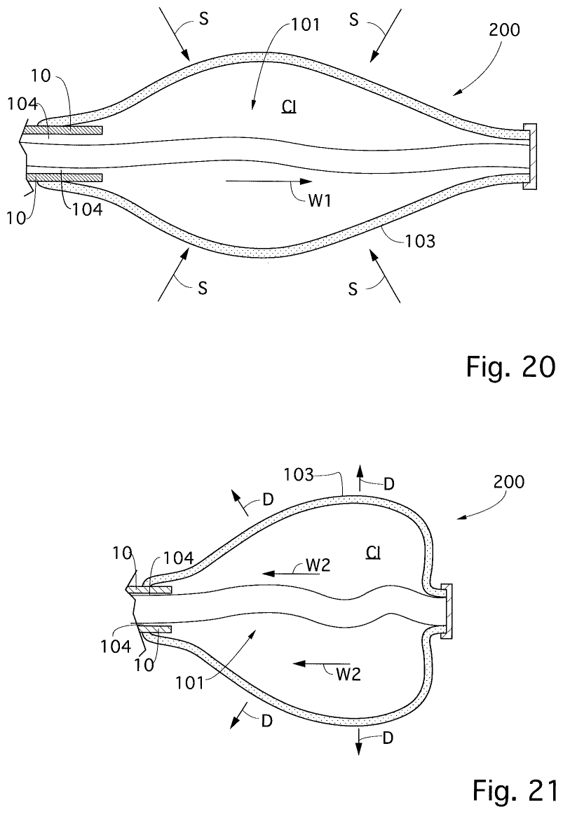

With reference to FIGS. 20 and 21, in which, for simplicity, the same reference numbers have been kept in order to indicate elements that are identical to the previous drawings, another embodiment of a balloon-shaped body can be seen, indicated with 200.

Also in this version, the balloon-shaped body 200 has an internal core 101, an external surface 103 and an internal chamber CI into which a fluid is intended to be introduced through the annular passages 104.

Also in this version, the length of the internal core 101 is modifiable to allow the balloon-shaped body 200 to follow the systole S and diastole D movements of the left ventricle VS in which the cardiovascular device according to the invention is intended to be implanted.

In this version, the modification means consist of the same sinusoid conformation of the central core 101, as can be seen in the drawings, which shortens during the diastole phase and lengthens during the systole phase.

Also in this version, the external surface 103 is kept constantly in sealed contact with the internal wall 5 of the left ventricle VS; this internal wall 5 is not shown for simplicity of representation.

Also in this version, the ends of the external surface 103 of the balloon-shaped body 200 are attached in a sealed manner respectively to the segment of duct 10 and to the core 101, preferably by means of gluing.

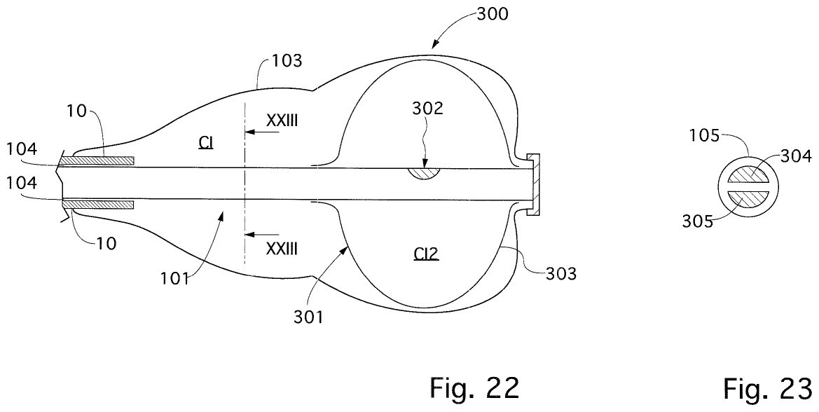

With reference to FIGS. 22 and 23, in which, for convenience, common elements with the previous drawings are indicated with the same reference numbers, another version of a balloon-shaped body, indicated with 300, can be seen.

Also in this version of the cardiovascular device according to the invention, there are provided the expandable external surface 103 of the balloon-shaped body 300, the internal core 101, the annular passage 104, controlled by the valve means 13 (not shown) and the segment of duct 10.

In the internal chamber CI defined in the balloon-shaped body 300 there is disposed a second balloon-shaped body 301, substantially concentric with the balloon-shaped body 300, and also able to expand or contract according to the movements of the left ventricle VS.

In detail, the core 101, also in this version, consists of the tubular element 105, therefore axially hollow, inside which a fluid can be fed, for example a saline solution, intended to occupy a second internal chamber CI2 defined in the balloon-shaped body 301, passing through at least one feed aperture 302 obtained in the tubular element 105 in correspondence with the second chamber CI2.

The second balloon-shaped body 301 also has its own elastically expandable or contractable external surface 303.

As in the previous versions, the ends of the balloon-shaped body 300 are attached in a sealed manner, preferably by means of gluing, to the duct segment 10 at one end and to the core 101 at the opposite end.

On the latter, the ends of the second balloon-shaped body 301 are also attached in a sealed manner, and also preferably by means of gluing.

As can be seen in detail in FIG. 23, the cross-section of the tubular element 105 comprises two separate ducts, indicated respectively with 304 and 305, one of which is intended for the passage of fluid in order to expand the second balloon-shaped body 301, while the other is intended for the passage of a possible guide wire conventionally used to position the cardiovascular device 1 inside the left ventricle VS, passing through a blood vessel of a patient.

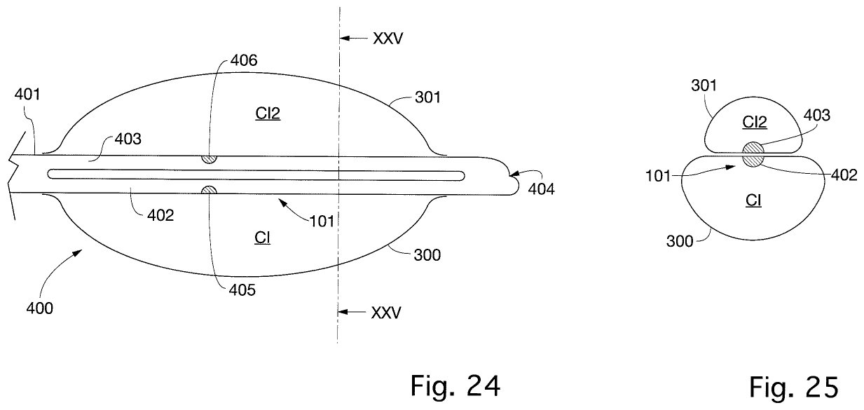

With reference to FIGS. 24 and 25, another variant of the balloon-shaped body can be seen, indicated in this case with 400.

Similarly to the version shown in FIGS. 22 and 23, also in this alternative embodiment there are provided a first balloon-shaped body 300 and a second balloon-shaped body 301, however attached to the central core 101 external to each other and disposed on opposite sides with respect to the central core 101.

The central core 101 also has a different conformation and consists of a tubular body 401 which includes two parallel channels 402 and 403 which are separated from each other for almost the entire length, but which join in reciprocal confluence in an end zone 404.

Both parallel channels 402 and 403 are provided with apertures 405 and 406 intended for the passage of at least one fluid used to expand or contract, elastically and alternatively, the two balloon-shaped bodies 300 and 301 following the systole and diastole movements of the left ventricle VS.

As can be seen in FIG. 24, the ends of the two balloon-shaped bodies 300 and 301 are attached in a sealed manner, preferably by means of gluing, respectively to the duct 402 and to the duct 403.

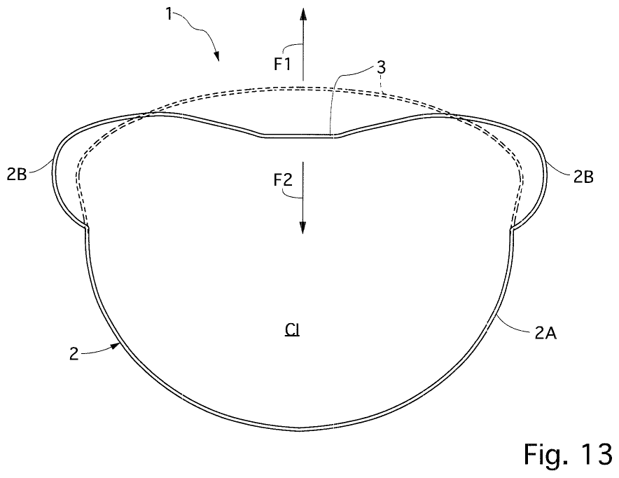

The functioning of the cardiovascular device according to the invention in the embodiments shown in FIGS. 1 to 16 is as follows: both in the case of percutaneous implantation, and also in the case of surgical implantation, the device 1 is prepared completely housed inside the axial cavity 16 of the tip 15, in a configuration completely folded back on itself in order to assume the smallest possible bulk.

In the case of percutaneous implantation, the surgeon makes a small access cut in the proximity of an artery, for example the femoral artery, and through this introduces the catheter 9 until the tip 15 reaches the inside of the left ventricle VS.

The surgeon holds the handpiece 14 with one hand and with the other acts on the plunger 17, thrusting the device 1 out of the axial cavity 16.

When this step is completed, the surgeon, by means of the syringe 8, injects, through the aperture 3A, into the internal cavity CI, a predetermined volume of saline solution, making the body 2 of the device 1 dilate, until it reaches a configuration in which the cap surface 2A and the edge 2B adhere in a sealed manner to the internal wall 5 and to the bottom 4 of the left ventricle VS, self-adapting to their profiles.

When the device 1 is correctly implanted, the surgeon detaches the catheter 9 from the aperture 3A, more precisely from the valve means 13 that control it and retracts it until it is completely removed from the artery in which it has been made to slide.

The device 1, since it is completely flexible, after implantation follows the cyclic contractions and dilations of the wall 5 of the left ventricle VS and, in particular when the systole phase of the latter occurs, the mobile portion 3 is everted toward the inside of the left ventricle VS, thrusting the blood accumulated inside it toward the aorta AO, while in the diastole phase the mobile portion 3 is released toward the bottom 4 allowing a new filling of blood in the left ventricle VS.

In the version for surgical implantation, the surgeon first proceeds to perform an incision on the patient's chest in the proximity of the heart C and makes an access cut also in the apical zone of the bottom 4 of the left ventricle VS.

Through this cut, the surgeon introduces the tip 15 of the catheter 9 and with the plunger 17 expels it from the internal cavity 16, releasing it in the left ventricle VS.

When the device 1 is correctly positioned, the surgeon injects the saline solution into the internal cavity CI with the syringe 8, passing through the one-way valve means 13, making the device 1 assume the dilated configuration until the cap portion 2A of the body 2 and the edge 2B adhere in a sealed manner to the internal wall 5.

The correct positioning of the device 1 inside the left ventricle VS allows, in this version, to keep the segment of duct 10 extended toward the outside of the apical zone of the heart C for a length sufficient to fit the split pin 11 over it, which in this way tightens between it and the body 2 the portion of cardiac tissue that forms the bottom 4 and on which the radial elements 12 are then folded in order to hold it.

After the implant, the surgeon sutures the cuts made and the device 1 begins its function, following the cyclical sequences of systole and diastole of the heart, as described above, acting as a pump and simultaneously reducing the internal volume of the left ventricle VS.

The person of skill will understand that the dimensions of the device 1 can be modified as needed, that is, according to the volume part of the left ventricle VS that the surgeon wishes to reduce.

The cardiovascular device in the other embodiment versions shown in from FIGS. 17 to 25 and which comprise one or two balloon-shaped bodies, can be implanted in a left ventricle VS as already described for the previous versions and the difference with these consists in the conformation of the balloon-shaped body used to occupy and occlude an apical part of the ventricle.

In detail, in these other embodiment versions, the balloon-shaped body generally comprises the central core 101 which on the one hand, in certain cases, has the function of allowing expansions and contractions of the external surface 103 to follow the systole and diastole movements of the heart C, and on the other hand in some cases has the function of transporting and feeding a fluid, typically a saline solution, which has to occupy the internal chamber or chambers CI and/or CI2 in the case of a double balloon-shaped body.

The central core 101 is elastically deformable in the axial direction in such a way as to allow guided expansions and contractions of the balloon-shaped body and, consequently, shortenings and lengthening of the central core.

In detail, in the version visible in FIGS. 17-19, the spring element 106 cyclically contracts in the diastole phase, loading and recalling the external surface 103, creating a blood suction phase inside the left ventricle VS, and, subsequently, it extends in the systole phase, unloading and thrusting, with the external surface 103, the blood suctioned by the left ventricle VS toward the aorta AO.

The person of skill will understand that the difference in length that the central core 101 can assume between the two configurations, contracted and extended, can be predetermined a priori during the manufacturing step of the cardiovascular device 1.

Similarly to the version shown in FIGS. 17-19, also in the one shown in FIGS. 20-21 the dynamic function of suction and thrust of the blood is the same, with the only difference being that the lengthening and shortening of the central core 101 are determined by the sinusoidal structure thereof and by its high elastic deformability that allows a contraction or a distension of the bends that form the sinusoid.

The movements of expansion and contraction of the external surface 103 perform, as mentioned, the phases of suction of the blood inside the left ventricle VS and of thrust toward the aorta AO.

In the embodiment version shown in FIGS. 22 and 23, the central core 101 has a fixed length and the cyclical movements of expansion and contraction of the external surface 103 of the balloon-shaped body 300 are obtained by feeding a saline solution both into the internal chamber CI and also into the second internal chamber CI2 of the second balloon-shaped body 301, disposed inside the balloon-shaped body 300.

The feed of the saline solution into the internal chamber CI occurs through the annular passage 104 regulated by the valve means 13, while the feed into the second chamber CI2 occurs through the axial cavity of the central core 101, which is also regulated by the valve means 13, and the aperture 302 obtained in the latter.

It should be noted that in this embodiment version of the cardiovascular device 1, the saline solution passes in one of the two ducts 304 or 305 obtained in the central core 101, while the other is intended for the passage inside it of a conventional guide wire typically used by operators to correctly position the cardiovascular device 1 inside the left ventricle VS.

With reference to the other embodiment of the balloon-shaped body 400 shown in FIGS. 24 and 25, the dynamic function of expansion and contraction of the balloon-shaped body 300 and of the second balloon-shaped body 301 is the same as the version shown in the previous FIGS. 22 and 23, with the difference being that the two balloon-shaped bodies are external and separate from each other and are individually fed with a saline solution through a respective channel 402 and 403, defined inside the central core 101, and the respective apertures 405 and 406.

The volumes of the two balloon-shaped bodies 300 and 301 may be the same or different in order to better adapt to the morphology of the internal wall 5 of the left ventricle VS.

It has been verified that the invention achieves the intended purposes.

The invention is susceptible to modifications and variants, all of which are within the scope of the inventive concept.

Furthermore, all the details can be replaced with other technically equivalent elements.

In their practical embodiment, any other materials, as well as shapes and sizes, can be used according to requirements, without departing from the main field of protection of the following claims.

1. A cardiovascular device (1) designed for a cardiac cavity (VS) which has a volume and wherein blood flows, which is bounded by walls (5) and which has a greater longitudinal dimension and a smaller transverse dimension, said device comprising:
a diaphragm assembly designed to be inserted into said cardiac cavity (VS) substantially transverse to said greater longitudinal dimension so as to reduce said volume, said diaphragm assembly having a peripheral edge (2B) sealingly engageable with said walls (5) and being alternately driven between an active blood thrust position and an inactive position, said diaphragm assembly that includes said peripheral edge (2B) being at least partially deformable in response to contractions of said walls (5);
characterized in that said diaphragm assembly comprises:
a balloon-shaped elastic body (2; 100, 200; 300, 400) which has an external surface (2A; 103) which defines and encloses an internal chamber (CI) and which is modifiable alternatively between a position of minimum bulk in said inactive position and an everted position of maximum bulk in said active position, and vice-versa;
at least one mobile portion (3; 3′) of said external surface (2A; 103) which is transverse/diagonal with respect to said greater longitudinal dimension and which is peripherally surrounded by said peripheral edge (2B);
at least one aperture (3A) for access from the outside to said internal chamber (CI).

2. The device according to claim 1, wherein said balloon-shaped body (2; 100, 200; 300, 400) has stiffening zones (6) of said external surface (2A) alternatively disposed alternated with more flexible zones (7).

3. The device according to claim 1, wherein said peripheral edge (2B) comprises a closed ring profile made of an elastically flexible material and everted with respect to said external surface (2A; 103).

4. The device according to claim 1, wherein said balloon-shaped body (2; 100, 200; 300, 400) comprises a segment of duct (10) for connection between said internal chamber (CI) and the outside.

5. The device according to claim 1, wherein said access aperture (3A) is equipped with valve control means (13), alternatively selectable in an open position or in a closed position.

6. Kit for dividing a cardiac cavity (VS), characterized in that it comprises:
a cardiovascular device (1) which has a balloon-shaped elastic body (2; 100, 200; 300, 400) which has an external surface (2A; 103) which defines and encloses an internal chamber (CI) and which is mobile alternately between a folded position of minimum bulk and an everted position of maximum bulk, and vice-versa;
at least one mobile portion (3; 3′) of said external surface (2A; 103) which is transverse/diagonal with respect to said greater longitudinal dimension and which is peripherally surrounded by a peripheral edge (2B);
at least one aperture (3A) for access from the outside to said internal chamber (CI);
a catheter (9) which has a housing tip (15) for internally receiving said cardiovascular device (1) in a folded configuration for introduction into a cardiac cavity (VS) and a plunger (17) for expelling said cardiovascular device (1) from said housing tip (15);
a syringe (8) for injecting a volume of fluid into said internal chamber (CI).

7. The kit according to claim 6, wherein it further comprises at least one split pin (11) which can be associated with said balloon-shaped elastic body (2; 100, 200; 300, 400) outside said cardiac cavity (VS).

8. A balloon-shaped elastic body (2; 100, 200; 300; 400) intended for a cardiovascular device (1) able to be implanted in a cardiac cavity (VS) which has a volume and wherein blood flows, which is bounded by walls (5) and which has a greater longitudinal dimension and a smaller transverse dimension, said elastic body having an external surface (103) which defines and encloses an internal chamber (CI, CI2) and which is modifiable alternatively between a position of minimum bulk in said inactive position and an everted position of maximum bulk in said active position, and vice-versa, characterized in that said balloon-shaped elastic body comprises an internal core (101) which passes through said internal chamber (CI, CI2) in a hermetically sealed manner and extends between a distal end and an apical end and which has an axial length able to be modified elastically with modification means between a dilated position of said external surface (2A; 103) and a contracted position of said external surface (2A; 103).

9. The balloon-shaped elastic body according to claim 8, wherein said modification means are selected from a spring element (106), a sinusoid conformation of said internal core (101).

10. The balloon-shaped elastic body according to claim 8, wherein said central core (101) comprises a tubular body (105).

11. The balloon-shaped elastic body according to claim 8, wherein it comprises a balloon-shaped body (100; 200; 300; 400) and a second balloon-shaped elastic body (301).

12. The balloon-shaped elastic body according to claim 11, wherein said second balloon-shaped elastic body (301) is located internally or externally to said balloon-shaped elastic body (300).

13. The balloon-shaped elastic body according to claim 8, wherein said internal chamber (CI) and second internal chamber (CI2) have volumes selected from equal volumes or different volumes.

14. The balloon-shaped elastic body according to claim 10, wherein said tubular body (105) comprises at least two internal ducts (304, 305; 402, 403) equipped with apertures (302; 405, 406) for connection with said internal chamber (CI) and second internal chamber (CI2).
